(19) United States
(12) Patent Application Publication
(10) Pub. No.: US 2022/0266296 A1
(43) Pub. Date: Aug. 25, 2022

(54) Methods of Photografting to Surface Metal Oxides

(71) Applicant: Orthobond Corporation, Princeton, NJ (US)

(72) Inventor: Jordan Katz, Short Hills, NJ (US)

(21) Appl. No.: 17/182,795

(22) Filed: Feb. 23, 2021

(51) Int. Cl. B05D 3/06; B05D 1/02; A61L 27/04; A61L 27/06; A61L 27/34; A61L 27/54

(52) U.S. Cl. CPC B05D 3/067; B05D 1/02; A61L 27/042; A61L 27/06; A61L 27/34; A61L 27/54; B05D 2202/10; B05D 2202/35; A61L 2300/208

(57) ABSTRACT

The present invention relates to methods of photocatalyzed bonding of compounds directly to surface metal oxides and the articles obtained therefrom. The methods for preparing an article having a compound bonded to a metal oxide comprise applying a compound that includes a photograftable moiety to the surface of the article; and applying light to the compound.

### FIELD OF THE INVENTION

The present disclosure relates to photocatalyzed bonding of compounds directly to surface metal oxides, including methods of UV-photocatalyzed bonding of compounds containing a photograftable moiety to surface metal oxides and articles having such compounds covalently bound to metal oxides present on surfaces thereof.

### BACKGROUND

Surface modification to impart desired or improved characteristics to articles of manufacture is among the primary applications of industrial chemistry. Classic examples include galvanized steel and non-stick cookware. An exemplary application of particular need and commercial interest is antimicrobial surface modification for prevention and control of infection associated with articles of manufacture that are used, for example, as medical devices and in particular, medical implants. Typically, control of infection is sought by the topical application of disinfectants, antiseptics, antimicrobials and the like to surfaces likely to be contacted by infectious agents. Common disinfectants only have a short-term effect and need to be reapplied constantly.

Antibiotics can be administered to stop infection in individuals. However, such administration is not always effective. Numerous medical applications, including orthopedic, trauma, spine and general surgery applications, where the potential for infection is a serious concern, are not amenable to simple application of an antiseptic or treatment with antibiotics. For example, infection can be a devastating complication of a total joint arthroplasty (TJA). While some infections may be treated by antibiotic suppression alone, more aggressive therapies, such as two-stage re-implantation, are often required. TJA infections occur when bacteria colonize the surface of the implant. These species then form a resistant biofilm on the implant surface, which nullifies the body's normal antibody response.

External fixation devices provide temporary and necessary rigid constraints to facilitate bone healing. However, patients risk pin-tract infection at the site extending from the skin-pin interface to within the bone tissue. Such complications can result in sepsis and osteomyelitis, which could require sequestrectomy for correction. Even the most stringent pin-handling and post-procedure protocols have only a limited effect. Studies have shown that such protocols do not reduce the chance of infection.

In minimally-invasive spine fusions, pedicle screws are first implanted in the bone of the vertebrae, and then rods are fixed into the heads of the screws to immobilize and stabilize the affected segments. Screws and rods pass through the patient's skin into the spine space via a cannulated channel. As in external fixation, screws and rods are also prone to pin-tract infections; due to the implants' exposure to the operating room environment and pathway through the skin, the chance of contacting and/or passing harmful bacteria is greatly increased.

Catheters and shunts are placed in any number of body cavities and vessels to facilitate the injection, drainage or exchange of fluids. Infections are common in catheter placements and are largely dependent on how long the patient is catheterized.

There is a need for anti-infective surfaces that may be employed in locations particularly susceptible to hosting infectious agents, such as public places, common areas of buildings, fixtures and the like. Moreover, there is a need for articles and materials with anti-infective surfaces, such as medical devices including implants, screws, rods, pins, catheters, stents, surgical tools and the like which could prevent infections by proactively killing pathogens that attempt to colonize the device surface both pre- and post-operatively.

The Applicant and others have heretofore developed a number of anti-infective surface treatments and processes for producing same. These generally involve attachment of an intermediate or bridge layer to a substrate surface, and subsequent bonding of anti-infective compounds to the intermediate layer to form the modified anti-infective surface. A need persists for faster, cheaper, more widely applicable methods of surface modification.

As described below, Applicant has discovered methods of surface modification, such as binding of anti-infective compounds, directly to an oxide layer of a substrate in a single step. Such methods, and the resulting modified surfaces, were unexpectedly found to exhibit anti-infective efficacy at least equivalent to prior art surface modifications while improving cost and throughput and minimizing, for example, exposure to exogenous chemical compounds.

### SUMMARY

The present disclosure relates to photocatalyzed bonding of compounds to surface metal oxides, including methods of covalently bonding compounds directly to surface metal oxides, and articles prepared thereby. The compounds can be selected to provide or improve a desired characteristic of the surface, such as antimicrobial activity. As such, in certain exemplary embodiments of the present disclosure, articles having antimicrobial compounds directly bound to their surfaces (without resort to, for example, an intervening surface layer for binding of such compounds) are provided.

In one aspect of the present disclosure, articles comprising a surface with a metal oxide and photograftable compound covalently bonded (as a radical) to the surface metal oxide are provided. In a related aspect, methods of preparing such articles are provided. In certain embodiments, the methods comprise the steps of applying a photograftable compound to the surface and subsequently applying ultraviolet (UV) light to form a covalent bond to the metal oxide. In certain embodiments, applying the compound comprises spraying the compound onto the surface (i.e., spray application).

In certain embodiments, the article comprises a metal substrate, such as a substrate comprising a transition metal. In some such embodiments, the substrate comprises at least one metal selected from the group consisting of titanium, iron, chromium, nickel, tantalum, zirconium and magnesium. In certain embodiments, the metal substrate is titanium. In certain embodiments, the metal substrate is an alloy. In some such embodiments, the alloy is selected from the group consisting of a titanium alloy, a stainless steel alloy, or a cobalt chrome alloy.

In additional and alternative embodiments, the metal oxide is synthetic, as described below. In some such embodiments, the metal oxide is a transition metal oxide, a post-transition metal oxide, or an alkaline earth metal oxide. For example, in certain embodiments, the metal oxide is a titanium oxide, a zirconium oxide, a magnesium oxide, or an aluminum oxide.

One aspect of the present disclosure concerns compounds suitable for use with the methods and articles disclosed herein. Suitable compounds include organic compounds containing a UV-photograftable moiety, as described below. For example, in certain embodiments, the compound contains a moiety selected from the group consisting of acryl or methacryl (collectively referred to herein as (meth)acryl) moieties and derivatives thereof, vinyl groups, epoxides, and dienes. In certain embodiments, the compound contains a (meth)acryl group or derivative thereof.

Another aspect of the present disclosure is directed to covalent modification of surface metal oxides with compounds which are photograftable and anti-infective, i.e., compounds containing a UV-photograftable moiety and an anti-infective moiety. In certain embodiments, the anti-infective moiety is a quaternary pyridinium, quaternary ammonium, or quaternary phosphonium moiety.

For example, in certain embodiments, the compound is selected from the group consisting of (meth)acryloyloxydodecylpyridinium salts, (meth)acryloyloxyhexadecylpyridinium salts, (meth)acryloyloxydecyltriethylammonium salts, 4-hexadecyl(meth)acryloyloxyethylpyridinium salts, (meth)acryloyloxyethylhexadecylbipyridinium salts, (meth)acryloyloxydodecyltrimethylphosphonium salts, (meth)acryloyloxyoctadecyltriethylphosphonium salts, 4-(meth)acryloyloxyethyldodecylpyrldinium salts, di(4-vinylbenzyl)hexadecylmethylammonium salts, di((meth)acryloyloxyethyl)dodecylmethylammonium salts and (meth)acryloyloxyethyl(4-N-hexadecylpyridinylmethyl) succinate halides. In certain embodiments, the compound is selected from methacryloyloxydodecylpyridinium bromide (MDPB), diallyldimethylammonium chloride (DADMAC), methacryloyloxyhexedecylpyridinium chloride (MHPC), 4-hexadecyhnethacryloyloxyethylpyridinium chloride (HMPC),
methacryloyloxyethylhexadecylbipyridinium dichloride (MHBP),
methacryloyloxyoctadecyltrimethylphosphonium chloride (DMPC),
methacryloyloxyoctadecyltriethylphosphonium acetate (OEPA), 4-methacryloyloxyethyldodecylpyridinium chloride (MEDP), di(4-vinylbenzyl)hexadecylmethylammonium methyl sulfate (VHMS),
di(methacryloyloxyethyl)dodecylmethylammonium chloride (DDMC) and
methacryloyloxyethyl(4-N-hexadecylpyridinylmethyl) succinate bromide (BMPS).

In another related aspect of the present disclosure, medical implants having an anti-infective compound covalently bonded to a surface metal oxide, and methods of preparing same, are provided. In certain embodiments, the methods comprise applying (e.g., spray-applying) an organic compound comprising a UV-photograftable moiety and an anti-infective moiety to a surface of a medical implant and subsequently applying UV light to the compound. In certain embodiments, the anti-infective moiety is a quaternary pyridinium, quaternary ammonium, or quaternary phosphonium moiety. In some exemplary embodiments, the compound is MDPB.

The substance and scope of the methods and articles disclosed herein is described and understood more fully with reference to the following.

### BRIEF FIGURE DESCRIPTION

FIG. 1 provides representative Fourier transform infrared spectra for each of a stainless steel coupon sprayed with MDPB and subject to UV photocatalysis and a stainless steel coupon sprayed with MDPB and subject to UV photocatalysis with prior deposition of vinyl phosphonate by aqueous anodization, as described in Example 2. Strong characteristic alkyl and ester peaks are apparent at ˜2900 cm⁻¹ and 1750 cm⁻¹, respectively.

### DETAILED DESCRIPTION

The present disclosure is directed to methods of modifying surfaces by directly bonding compounds to metal oxides present on the surfaces, and articles having surface metal oxides with compounds directly bound thereto. Various aspects of the disclosure are discussed in additional, nonlimiting detail below.

### Surfaces

Articles according to the present disclosure comprise at least one surface comprising a metal oxide and a compound comprising a compound with a UV-photograftable moiety covalently bonded thereto. For example, in certain embodiments, a medical or dental implant comprising a surface having a metal oxide is provided, wherein a compound is covalently bonded to the metal oxide overlayer, the compound comprising (as covalently bound to the overlayer) a radical of a photograftable moiety and an anti-infective moiety.

In certain embodiments, the article comprises, in whole or in part, a metal substrate. Accordingly, in some embodiments, the surface having the metal oxide overlayer is itself a metal (or metal alloy). Substrates suitable for use with the present invention include any metal or metalloid capable of forming a “native” surface metal oxide (or “passivation layer”) and those capable of being provided with a surface metal oxide coating by conventional techniques.

The surface metal oxide is present at the surface of the article. For example, in some embodiments, the surface metal oxide is present as a generally continuous layer several nanometers in depth at the interface of the substrate and the environment.

Suitable substrates, surfaces, and/or metal oxides include those comprising at least one transition metal, post-transition metal, or alkaline earth metal. For example, in certain embodiments, the surface consists of or comprises titanium, chromium, nickel, molybdenum, tantalum, zirconium, magnesium, vanadium, zinc, niobium, tin, or aluminum. Further by way of example, in certain of these and other embodiments, the substrate comprises a metal alloy, such as a titanium alloy, a stainless steel, or a cobalt chrome alloy. With reference to the Examples below, in some embodiments the article or surface comprises titanium, a titanium alloy, or stainless steel.

### Surface Metal Oxides

As used herein, the term “surface metal oxide” refers to metal oxides covalently bound to the surface of the article, including corresponding native oxide (or passivation) layers. As such, the term includes metal oxides often present on metal and metal alloy surfaces under ambient environmental conditions, as well as synthetic surface metal oxide layers as described below.

For example, stainless steel alloys are insulated from extensive iron rusting by the presence of a threshold content of chromium, which, when oxidized at the surface, forms a thin chromium (III) oxide layer which functions as a passivating barrier against further corrosion. Similarly, as described in U.S. Pat. No. 7,507,483, hereby incorporated by reference in its entirety, exposure of a clean surface of titanium materials to oxygen results in the spontaneous formation of surface titanium oxides which protect the underlying material from further chemical reaction.

In corresponding embodiments, the surface metal oxides will comprise oxides of the metal or metals constituting the surface of the article substrate. For example, the metal oxide overlayer of a Ti-6Al-4V titanium alloy may include oxides of titanium, aluminum, and vanadium, while the metal oxide overlayer of a stainless steel alloy may include oxides of chromium, iron, nickel, molybdenum, and niobium, among others. Without limitation, the metal oxide overlayer can comprise one or more oxides of a period 4 or period 5 transition metal, aluminum, tin, or tantalum, or magnesium.

### Surface Metal Oxide Modification and Synthesis

In certain embodiments according to the present disclosure, the surface of the article is treated to modify its surface metal oxide character, such as by increasing the relative number of surface metal oxides available for binding. For example, the depth, density, and/or composition of the overlayer can be modified by heating the article, treating the surface of the article with an acid or base, anodization, or gas plasma surface treatment, as known in the art. Such treatments can increase the density of surface metal oxides and/or metal oxide hydroxylation. Accordingly, in certain embodiments, the article or surface is subject to one or more treatments to modify the metal oxide overlayer prior to compound coating and photografting.

In related and alternative embodiments according to the present disclosure, the surface metal oxides are synthetically imparted by chemical treatment of the surface. For example, in some embodiments, metal and non-metal (e.g., polymeric) surfaces are treated to incorporate a synthetic metal oxide overlayer, which is subsequently coated with a compound and subject to applied UV light as disclosed herein.

For example, as disclosed in Applicant's U.S. Patent Publication No. 2018/0103643, hereby incorporated by reference in its entirety, synthetic metal oxide surfaces may be imparted on metal, metalloid or non-metal surfaces by, for example, reacting a surface with a metal alkoxide, optionally followed by full or partial hydrolysis. In certain embodiments, the surface of a polymer substrate is modified to possess a metal oxide overlayer by bonding a metal alkoxide layer thereto, followed by pyrolysis or hydrolysis of the metal alkoxide to yield a continuous surface-bound metal oxide overlayer. Suitable polymers include polyamides, polyurethanes, polyesters, polyketones, polyethers, polyimides, aramides, polyfluoroolefins, epoxies, silicones or composites containing same. For example, substrates that contain acidic protons, such as —OH or —NH groups, are functionalized by their reaction with Group IV alkoxides. Alternatively, in certain embodiments, surfaces comprising native metal oxides can be modified to comprise synthetic metal oxides, which may, for example, provide a more favorable substrate for photografting as disclosed herein.

Alkoxides of transition metals are particularly useful for the synthesis of surface metal oxides in this manner. Preferred metals include Zr, Al, Ti, Hf, Ta, Nb, V and Sn, with suitable metal oxides including, without limitation, tantalum pentethoxide, titanium tetra-t-butoxide and zirconium tetra-t-butoxide.

### Compounds

In methods according to the present disclosure, photograftable compounds are covalently bonded to surface metal oxides by application of UV light. Corresponding articles comprise photograftable compound radicals covalently bonded to surface metal oxides. In preferred embodiments, the photograftable compounds are organic.

As used herein, the term “photograftable” refers to the ability of a compound to form a stable covalent bond with a surface metal oxide by photocatalysis. Accordingly, a photograftable compound according to the present disclosure is a compound capable of photocatalyzed (light-catalyzed) bonding to a surface metal oxide. Such compounds contain at least one functional group or chemical moiety (“photograftable moiety”) capable of photocatalyzed radical formation or, as discussed below, sufficiently nucleophilic to bond with photocatalysis-generated reactive surface metal oxide species.

In certain embodiments, including presently preferred embodiments, the compounds comprise a UV-photograftable moiety—i.e., a chemical group capable of UV light-catalyzed radical formation or bonding a UV light-catalyzed reactive surface metal oxide species. Visible and infrared light are also suitable in certain embodiments, particularly with the use of a photoinitiator to promote radical formation and/or extended light application.

Preferred compounds according to the disclosure further comprise at least one moiety that modifies a surface characteristic of the article. For example, in certain embodiments of the present disclosure, the compound comprises a UV-photograftable moiety and an anti-infective moiety such as a quaternary ammonium, pyridinium, or phosphonium moiety.

UV-photograftable moieties according to the present disclosure include those moieties which form a covalent bond with a surface metal oxide when a compound containing such moiety is applied to or in contact with a surface metal oxide, such as in a coating (as described in further detail below), and the compound and/or surface metal oxide is exposed to applied UV light. UV-photograftable moieties also include moieties which form a covalent bond with a metal oxide overlayer in the presence of a photoinitiator under like conditions.

UV-photograftable moieties according to the present disclosure include, for example, acrylate and methacrylate moieties, acrylamide and methacrylamide moieties, thioacrylate and thioacrylamide moieties, and derivatives thereof, referred to collectively herein as acryl moieties Generally, under appropriate conditions (known to or readily ascertained by a person of ordinary skill in the art) all chemical groups that can participate in radical polymerization are suitable for use as UV-photograftable moieties , UV-photograftable moieties include, for example, radical-forming functional groups present in photopolymerizable monomers. Such moieties are known in the art, and frequently contain, for example, one or more ethylene (ethylenic unsaturation) groups, conjugated π systems, strained ring systems, and/or polar groups. In certain embodiments, the compounds comprise a UV-photograftable moiety selected from the group consisting of acryl moieties, vinyls, vinyl ethers, epoxides, and diallyls,

In certain embodiments in accordance with the present disclosure, the compound further comprises an anti-infective moiety. For example, in some embodiments, the compound comprises a UV-photograftable moiety and a quaternary pyridinium salt, a quaternary ammonium salt, or a quaternary phosphonium salt.

Representative compounds comprising a UV-photograftable moiety according to the present disclosure include, without limitation, methacryloyloxydodecylpyridinium salts, methacryloyloxyhexadecylpyridinium salts, 4-hexadecylmethacryloyloxyethylpyridinium salts, methacryloyloxyethylhexadecylbipyridinium salts, 4-methacryloyloxyethyldodecylpyridinium salts and methacryloyloxyethyl(4-N-hexadecylpyridinylmethyl) succinate halides. For example, suitable compounds include methacryloyloxydodecylpyridinium bromide (MDPB), methacryloyloxyhexedecylpyridinium chloride, 4-hexadecylmethacryloyloxyethylpyridinium chloride, methacryloyloxyethylhexadecylbipyridinium dichloride, 4-methacryloyloxyethyldodecylpyridinium chloride, and methacryloyloxy-ethyl(4-N-hexadecylpyridinylmethyl) succinate bromide.

Suitable compounds further include methacryloyloxydecyltriethylammonium salts, di(4-vinylbenzyl)hexadecylmethylammonium salts and
di(methacryloyloxyethyl)dodecylmethylammonium salts diallyldimethylammonium chloride,
di(4-vinylbenzyl)hexadecylmethylammonium methyl sulfate,
di(methacryloyloxyethyl)dodecylmethylammonium chloride.

In certain embodiments, the compound is a quaternary phosphonium methacrylate or methacrylamide compound as disclosed in U.S. patent application Ser. No. 14/922,983, issued as U.S. Pat. No. 9,868,808, assigned to Applicant and hereby incorporated by reference in its entirety. In certain corresponding embodiments, the quaternary phosphonium compound is a radical of either of the following formulas, wherein n2 or n3 is between 1 and 50.

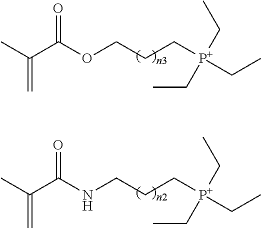

Thus, in certain embodiments, including the exemplary embodiments recited above, the compound comprises a (meth)acrylate or meth(acrylate) moiety and a quaternary pyridinium, ammonium, or phosphonium moiety.

In certain embodiments according to the present disclosure, the application of UV light results in oligomerization or polymerization of the compounds. For example, the present disclosure includes articles comprising a surface with a metal oxide overlayer and oligomeric or polymeric anti-infective compounds covalently bonded thereto. Such articles can be prepared by photopolymerization of polymerizable antimicrobial monomers by application of UV light as described herein.

Accordingly, methods of the present disclosure include those in which the compounds are polymerizable antimicrobial monomers, such as the quaternary pyridinium, ammonium, and phosphonium compounds provided above. For example, in certain embodiments, the compound is MDPB. Thus, representative methods include those comprising application of MDPB to a metal oxide overlayer of a surface followed by exposure to UV light.

### Photoactivation of Surface Metal Oxides

In additional and alternative embodiments according the present disclosure, bond formation between photograftable compounds and surface metal oxides is catalyzed at least in part by photoactivation of the surface metal oxides themselves. For example, and without limitation by theory, light absorption by metal oxides can disrupt electron ordering, resulting in the generation of reactive surface metal oxide species.

Thus, in certain embodiments, application of light (e.g., UV light) to the article catalyzes the formation of reactive surface metal oxide species, which species in turn bond to the photograftable compound. Generally, in such embodiments, the photograftable moiety is sufficiently nucleophilic to form a covalent bond with an electrophilic surface metal oxide species. Photocatalytic metal oxides are known in the art. Generally, transition metal oxides, including titanium dioxide, zinc oxide, and nickel oxide, are particularly suitable for photocatalyzed bonding as disclosed herein.

### Compound Coatings and Application

In methods according to the present disclosure, compounds are applied to the surface metal oxide of an article and covalently bound thereto by application of light. In currently preferred embodiments, the light is UV light. The compounds can be applied to the metal oxide overlayer by conventional means.

Generally, the compounds are applied to the metal oxide overlayer in solution, but can be applied neat if preferred. Suitable solutions will contain the compounds, solvents, and other components which may be removed from the metal oxide overlayer after application by evaporation or washing. For example, in certain embodiments, the compounds are applied in solution, such as in a suitable organic solvent optionally containing, for example, one or more photoinitiators, viscosity modifiers, agents to improve visibility, and/or other components to assist application.

The solution can be applied as a coating and dried prior to UV light application. For example, compounds can be applied by atomized spray application of a solution containing such compound, an organic solvent, and, optionally, a photoinitiator, or by brush-applying, wiping, or immersing an article in such solution. Compounds can be applied neat by the same techniques, or by, for example, electrostatic deposition, rubbing, polishing, or by electrochemical bath. Unevaporated components can be removed from the surface by washing after UV application.

After the compounds are applied to the metal oxide overlayer, and while the compounds are in contact with the metal oxide overlayer, UV light is applied. For purpose of the present disclosure, application of UV light refers to intentional UV light exposure, such as by placing in proximity to a UV light source (i.e., a lamp specifically purposed for emitting UV light), and excludes unintentional incidental exposure to background or ambient UV light.

UV light can be applied by conventional means. For example, in certain embodiments, articles having compounds in contact with a metal oxide overlayer on a surface thereof are placed in a UV chamber or UV ozone cleaner with nitrogen purging for a suitable duration of UV light exposure.

In certain embodiments, the photocatalytic UV light can be applied at a wavelength of about 370 nm or less, or about 300 nm or less, or about 254 nm or less. In additional and alternative embodiments, visible light is applied at a wavelength of about 700 nm or less. The light can be applied at an intensity of about 5 mW/cm² or less, or about 10 mW/cm² or less, or about 50 mW/cm² or less, or about 100 mW/cm² or less, or about 1000 mW/cm² or less. The surface of the substrate having the composition thereon can be irradiated with the UV light for about 5 minutes or less, or 10 minutes or less, or 15 minutes or less, or 20 minutes, or less, or 30 minutes or less, or 1 h or less, or about 2 h or less, or about 5 h or less, or about 10 h or less, or about 20 h or less, or about 30 h or less.

The polymer surface may be completely coated with a photoresist, and exposed to UV light through a mask. The areas exposed to the UV light can be developed and removed, leaving openings in the photoresist and access to the polymer surface in small areas.

Following UV light application, the article can be cleaned, such as by rinsing and/or sonication in suitable solvent(s). Repeated compound application and UV application cycles can be applied as desired.

### Articles

Any article with a surface having a surface metal oxide, including a metal oxide layer that is at least partially synthetic as described above, can be treated in accordance with the methods disclosed herein. As such, articles according to the present disclosure include, without limitation, consumer products, industrial equipment, components of infrastructure, medical devices, and dental implants. The surface characteristics of the articles treated as disclosed herein can be modified as desired based on selection of the compound(s) applied and covalently bound thereto via the metal oxide overlayer. By way of example and not limitation, depending on the compound(s) applied, article surfaces can be rendered more hydrophilic, hydrophobic, oleophilic, oleophobic, smooth, scratch-resistant, easy to clean, anti-reflective, and/or anti-infective.

For example, there is a need for anti-infective surfaces that may be employed in locations particularly susceptible to the colonization and transmission of infectious agents, such as in the healthcare setting, other public places, common areas of buildings, fixtures and the like. These protections on high-touch surfaces, fixtures, walls or other areas, would make transmission of viable pathogens less likely. The need for protection extends to other high-touch surfaces that are mobile, and can be transported from room-to-room and place-to-place, such as mobile device protection (antimicrobial screen protectors), keyboards, monitors and other control screens. Moreover, there is a need for articles and materials with anti-infective surfaces, such as medical devices including implants, screws, rods, pins, catheters, stents, surgical tools and the like which could prevent infections by proactively killing bacteria that attempt to colonize the device surface both pre- and post-operatively.

Present articles include those with surfaces likely to be colonized by microorganisms that impact human health, including human pathogens as well as other organisms such as those involved in food spoilage. These surfaces can be deployed in a healthcare, public, or private setting. Representative articles according to the present disclosure include, for example, laptop and cellular phone chassis, door handles, commuter handrails, faucets and taps, nuts, bolts, and washers, firearms, engine components, exhaust pipes, refrigerator doors, toys, and food and beverage containers, dental and orthopedic screws, and implantable replacement joints.

Present articles also include, for example, household articles such as cutting boards, sinks, utensils, counter tops, packaging, food storage containers, refrigerator parts, coolers and the like.

Present articles also include for example articles employed in businesses, hospital and/or nursing home environments such as walls, floors, bed-pans, sinks, and fixtures, and high-touch surfaces in public and private environments likely to be colonized with viable organisms, such as rails, door knobs, push panels, and the like.

Present articles also include high-touch surfaces such as mobile device touch screens and protectors, computers, laptops, keyboards, monitors, control panels screens for healthcare equipment, and the like. Present articles further include food- and beverage-contacting surfaces, such as food and beverage packaging, storage containers, shipping containers, and the like.

Present articles are for instance medical devices such as implantable or percutaneous medical devices. Medical devices include endoscopic, arthroscopic, laparoscopic, cardiac, cardiovascular, vascular, non-woven mesh, woven mesh, foam, cloth, fabric, orthopedic, orthopedic trauma, spine, surgical, drainage catheter, shunt, tape, meshes, rope, cable, wire, suture, skin and tissue staple, burn sheet, external fixation and temporary/non-permanent implant devices.

In certain embodiments according to the present disclosure, including presently preferred embodiments, at least one compound comprises a UV-photograftable moiety and an anti-infective moiety, such that the surfaces of articles treated in accordance with the present disclosure are rendered anti-infective. For example, in certain embodiments, the compound comprises a (meth)acrylate or meth(acrylamide) moiety and a quaternary pyridinium, ammonium, or phosphonium moiety. The quaternary pyridinium, ammonium, or phosphonium moiety is preferably but not necessarily present in ionizable form, such as a salt or halide.

In additional and alternative embodiments, including presently preferred embodiments, the article is a medical or dental device intended for at least temporary insertion or implantation in a human or animal. By way of example, and not limitation, in some embodiments, the article is a dental implant, such as a screw for fixation of an artificial tooth to the jaw, a surgical tool, such as a venipuncture needle, suture needle, scalpel, or forceps, a medical device, such as an implantable pacemaker, deep-brain stimulator, or stent, an orthopedic implant, such as a rod, screw, or plate for bone or spinal fixation, or a replacement joint implant, such as a replacement knee or hip device.

Thus, in certain embodiments, including presently preferred embodiments, the article is a medical implant, and a surface of the article is rendered anti-infective by covalently bonding a compound comprising a UV-photograftable moiety and an anti-infective moiety to a metal oxide overlayer of the surface by application of the compound to the surface and application of UV light thereto. In certain embodiments, the article is a medical implant and the compound is an anti-infective monomer comprising a (meth)acrylate or (meth)acrylamide moiety selected from the groups of anti-infective compounds recited above. For example, in certain embodiments, the article is a medical implant and the compound is MDPB.

The subject matter of the present invention is further described and enabled according to the following examples, which are representative and non-limiting.

### EXAMPLE 1

As disclosed in Applicant's U.S. Patent Publication No. 2018/0103643, incorporated by reference above, Applicant previously invented processes for preparing articles having functional surfaces by using aqueous anodization to deposit an organophosphorus surface layer and subsequently attaching functional compounds (e.g., MDPB) to the surface layer.

In an effort to optimize related processes for use with articles susceptible to formation of unreactive surface iron oxides during anodization, Applicant conceived the approach of coating such articles with a thin (˜50 μm) surface layer of a metal less susceptible to formation of unreactive oxides, such as titanium, by plasma vapor deposition (PVD), and undertook experiments to determine if such approach could provide improved functional surfaces prepared using aqueous anodization.

Initial procurement efforts identified a third-party service provider offering titanium nitride (TiN) PVD coatings. Stainless steel coupons PVD-coated with TiN were acquired and treated according to Applicant's aqueous anodization process by immersion in 15 wt % vinyl phosphonate at 15V for 30 seconds. Prior to MDPB coating (see below), Fourier transform infrared (FTIR) spectroscopy was conducted on TiN-coated coupons with and without phosphonate anodization to confirm the absence of potentially confounding absorption signals. These control spectra showed no clear absorbance signal as expected.

The coupons (TiN-coated coupons with and without phosphonate anodization) were then sprayed with a solution of MDPB in ethanol (1 g MDPB in 70 mL ethanol), with excess removed after spraying with a 10 μL pipette. The coupons were then placed in a UV chamber (Novascan PSDP-UV; λ=254 nm and 185 nm) on Aluminum foil, subject to nitrogen purge for 10 minutes, then subject to UV irradiation for 15 minutes with top and bottom lamps on. Following UV irradiation, the coupons were cleaned by washing with water, sonicating in water for 15 minutes, then sonicating in ethanol for 15 minutes.

After cleaning, Fourier transform infrared (FTIR) spectroscopy was conducted to determine the presence of MDPB on the surface of the coupons. Unexpectedly, the spectra of all coupons (i.e., MDPB-sprayed, UV-irradiation, TiN-coated coupons with and without phosphonate anodization) exhibited large alkyl and ester peaks, indicating the presence of MDPB.

Thus, corresponding coupons were prepared but subjected to extended cleaning to remove potentially confounding unbound residual MDPB. The coupons were sonicated for 3 hours in ethanol, 2 hours in phosphate buffered saline (PBS), another hour in ethanol, left in ethanol overnight, washed in ethanol, and finally dried under vacuum to 190 mTorr prior to FTIR spectroscopy. The spectra of the coupons subject to phosphonate anodization exhibited large alkyl and ester peaks. Surprisingly, however, the spectra of the control coupons not subject to phosphonate anodization also exhibited alkyl and ester peaks which, although smaller than those of the phosphate-anodized coupons, were nonetheless distinct. Since this suggested the possible presence of MDPB bound directly to the TiN coating, a competitive fluorescein binding assay was conducted to confirm the presence of quaternary ammonium ions (i.e., MDPB). The results, summarized in Table 1 below, confirmed the presence of MDPB on the surface of the nonanodized titanium nitride coated coupon.

TABLE 1

| Group | Abs | [M] | Total QA | QA/cm2 |
|---|---|---|---|---|
| (1) TiN | 0.001 | 1.30 E−8 | 5.16 E13 | 1.65 E13 |
| (2) TiN + MDPB | 0.024 | 3.12 E−7 | 1.24 E15 | 3.96 E14 |
| (3) TiN + Phos + MDPB | 0.429 | 5.57 E−6 | 2.21 E16 | 7.08 E15 |

In Table 1, the group designations refer to: (1) TiN-coated stainless steel coupons; (2) TiN-coated stainless steel coupons sprayed with MDPB and UV-irradiated; and (3) TiN-coated stainless steel coupons subject to phosphonate anodization and sprayed with MDPB and UV-irradiated. In this and subsequent tables, the Abs, [M], Total QA, and QA/cm2 column headings denote Absorbance, Molar Concentration, Quaternary Ammoniums, and Quaternary Ammoniums per square centimeter, respectively.

### EXAMPLE 2

The experiment of Example 1 was repeated with 4 groups: (1) stainless steel coupons with MDPB spray coating and UV photocuring (SS+UV MDPB; SS M); (2) stainless steel coupons subject to vinyl phosphonate electrodeposition and subsequent MDPB spray coating and UV photocuring (SS+electro vinyl phosphonate+UV MDPB; SS P+M); (3) titanium nitride PVD coated stainless steel coupons with MDPB spray coating and UV photocuring (SS−TiN+UV MDPB; TiN M); and (4) titanium nitride PVD coated stainless steel coupons subject to vinyl phosphonate electrodeposition and subsequent MDPB spray coating and UV photocuring (SS−TiN−electro vinyl phosphonate+UV MDPB; TiN P+M). All treatments were performed substantially as described for Example 1 above. 3 coupons per group were prepared.

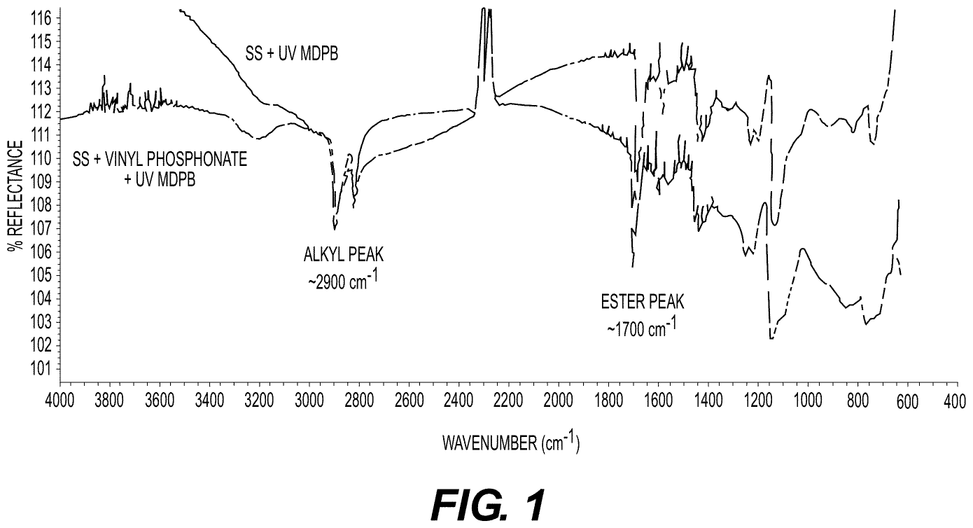

After treatment, 2 samples per group were cleaned by sonication for 2 h in PBS, 2 h in EtOH, 2 h in PBS, then 10 minutes in EtOH, and dried under vacuum to <200 mTorr for FTIR spectroscopy and fluorescence analysis. Representative spectra obtained for group 1 (SS+UV MDPB) and group 2 (SS+electro vinyl phosphonate+UV MDPB) coupons are provided in FIG. 1. Group 1 (SS+UV MDPB), group 2 (SS+electro vinyl phosphonate+UV MDPB), and group 4 (SS−TiN+electro vinyl phosphonate+UV MDPB) exhibited distinct alkyl and ester peaks at ˜2900 cm⁻¹ and ˜1750 cm⁻¹, respectively, indicating that MDPB remained bound to these samples after washing. Fluorescence data is provided in Table 2 below.

TABLE 2

| Group | Sample | Abs | [M] | Total QA | QA/cm2 | Avg | SD |
|---|---|---|---|---|---|---|---|
| SS control | | 0.006 | 7.79E−8 | 3.10E14 | 9.90E13 | | |
| TiN control | | 0.009 | 1.16E−7 | 4.64E14 | 1.48E14 | | |
| 1 (SS M) | 1 | 0.375 | 4.87E−6 | 1.94E16 | 6.19E15 | 5.57E15 | 8.63E14 |
| | 2 | 0.301 | 3.91E−6 | 1.55E16 | 4.96E15 | | |
| 2 (SS P + M) | 1 | 0.371 | 4.82E−6 | 1.92E16 | 6.12E15 | 6.52E15 | 5.60E14 |
| | 2 | 0.419 | 5.44E−6 | 2.16E16 | 6.91E15 | | |
| 3 (TiN M) | 1 | 0.017 | 2.21E−7 | 8.78E14 | 2.08E14 | 4.04E14 | 1.75E14 |
| | 2 | 0.032 | 4.16E−7 | 1.65E15 | 5.28E14 | | |
| 4 (TiN P + M) | 1 | 0.365 | 4.74E−6 | 1.88E16 | 6.02E15 | 6.64E15 | 8.75E14 |
| | 2 | 0.440 | 5.71E−6 | 2.27E16 | 7.26E15 | | |

In Table 2, the Avg and SD column headings denote, respectively, the average and standard deviation of the QA/cm2 measurements for the two samples per treatment group.

### EXAMPLE 3

1 coupon from each group above was preserved without FTIR or fluorescence analysis. These samples were subject to an additional cycle of washings and sonications as above, followed by incubation for seven days in PBS. Sample fluorescence was determined after PBS storage and is provided in Table 3 below. The fluorescence signal for the non-TiN treated stainless steel samples (group 1 and group 2), as well as the group 3 sample, were substantially identical to those observed for Example 2 above. The signal observed for the group 4 (SS−TiN+electro vinyl phosphonate+UV MDPB) was substantially reduced after washing and PBS incubation.

TABLE 3

| Sample | Abs |
|---|---|
| Control | 0.002 |
| Group 1 | 0.333 |
| Group 2 | 0.411 |
| Group 3 | 0.014 |
| Group 4 | 0.062 |

### Discussion

Surface modifications involving bonding to substrate surface oxide layers have heretofore been disclosed by Applicant and others. U.S. Pat. No. 7,507,483 to Schwartz et al., for example, discloses articles, including implantable devices, comprising surfaces with an oxide overlayer and a functionalized organophosphonic acid coating layer covalently bonded thereto. Applicant's U.S. patent application Ser. No. 15/785,789, published as U.S. Patent Publication No. 2018/0103643, discloses, inter alia, formation of surface oxide layer-bound organophosphorus layers by aqueous anodization. These techniques involve the application of a phosphonate “bridge” between surface metal oxides and compounds imparting modified surface characteristics, such as anti-infective compounds

In contrast to the foregoing and other prior art surface modification techniques, the methods of the present disclosure permit direct covalent modification of surface metal oxides with compounds of interest, such as anti-infective compounds, without resort to intervening (“bridge”) compounds or layers. Direct surface application and one-step bonding as disclosed herein provides significant advantages in scope, scale, speed and cost of surface modification relative to prior art techniques.

For example, and in contrast to comparable prior art surface modification techniques, including those noted above, the methods of the present disclosure can be performed “in the field” as well as in a laboratory or factory setting. Accordingly, in certain embodiments according to the present disclosure, previously installed articles are surface-modified. By way of example, and not limitation, previously installed fixtures that may act as potential fomites are surface-modified with an anti-infective compound to mitigate spread of pathogenic microorganisms. For example, in certain embodiments, MDPB is spray-applied to hospital door handles and UV irradiation is applied to the MDPB and handles via portable UV lamp to render the handles relatively anti-infective.

Single-step surface modification further obviates significant expenditures in time, labor, and materials. Prior art techniques generally require at least a day to apply, fix, dry, and finish intermediate compounds/layers, and the intermediate compounds (e.g., organophosphonic acids) themselves are expensive. The methods of the present disclosure are also amenable in many instances to spray application of the compound or compounds of interest, providing significant potential cost savings over comparable prior surface modification techniques. MDPD, for example, costs on the order of $30,000 per kilogram, and is applied in conventional surface modification techniques by dip coating in solution at a concentration of 5 mg/mL. In certain embodiments according to the present disclosure, MDPB is spray-applied at a concentration of 1 mg/mL, reducing material expenses as much as one-hundredfold relative to prior art methods.

Despite the significant advantages associated with the surface modification techniques disclosed herein, the Applicant is believed to be the first to observe and document the feasibility of durable UV-catalyzed covalent bond formation between photopolymerizable monomers and surface metal oxides. In particular, it was heretofore believed that to the extent, if any, that such bonds could arise, they would inherently be transient and unstable due to the intrinsic lattice structure and associated electron ordering of surface metal oxides.

In particular, and as described in the examples above, Applicant first observed the direct UV-catalyzed surface metal oxide bonding serendipitously, in experimental controls. As this observation was entirely unexpected, particularly in light of the relatively inert nature of titanium nitride, confirmatory experiments were conducted which demonstrated even more robust UV-catalyzed MDPB bonding with untreated (i.e., phosphonate uncoated) stainless steel. With the benefit of the present disclosure and the experiments described herein, a person of ordinary skill is readily able to identify working and suboptimal or non-working embodiments by routine optimization.

Based on the foregoing disclosure, working and non-working embodiments and modifications according to the present disclosure and the following claims are readily ascertainable by the person of ordinary skill. The subject matter of the present disclosure is further described and exemplified in the claims which follow.

1. A method of preparing an article having a compound covalently bonded to a metal oxide present on a surface of the article, the method comprising:
applying a compound comprising a photograftable moiety to the surface of the article; and
applying light to the compound.

2. The method of claim 1, wherein the photograftable moiety is a UV-photograftable moiety, and the light is UV light.

3. The method of claim 1, wherein the article comprises a metal substrate.

4. The method of claim 3, wherein the metal substrate comprises a transition metal.

5. The method of claim 3, wherein the metal substrate comprises a metal selected from the group consisting of titanium, iron, chromium, nickel, tantalum, zirconium and magnesium.

6. The method of claim 5, wherein the metal substrate is titanium, a titanium alloy, or a stainless steel alloy.

7. The method of claim 6, wherein the metal oxide present on the surface of the article is a titanium oxide, an iron oxide, or a chromium oxide.

8. The method of claim 1, wherein the metal oxide is synthetic.

9. The method of claim 1, further comprising a step of treating the surface to modify the metal oxide overlayer prior to application of the compound.

10. The method of claim 2, wherein the UV-photograftable moiety is selected from the group consisting of acryl moieties, vinyls, vinyl ethers, diallyls, dienes, and epoxides.

11. The method of claim 1, wherein the compound further comprises an anti-infective moiety.

12. The method of claim 11, wherein the anti-infective moiety is a quaternary pyridinium, quaternary ammonium, or quaternary phosphonium moiety.

13. The method of claim 12, wherein the compound comprises a moiety selected from the group consisting of a (meth)acryloyloxydodecylpyridinium salts, (meth)acryloyloxyhexadecylpyridinium salts, (meth)acryloyloxydecyltriethylammonium salts, 4-hexadecyl(meth)acryloyloxyethylpyridinium salts, (meth)acryloyloxyethylhexadecylbipyridinium salts, (meth)acryloyloxydodecyltrimethylphosphonium salts, (meth)acryloyloxyoctadecyltriethylphosphonium salts, 4-(meth)acryloyloxyethyldodecylpyridinium salts, di(4-vinylbenzyl)hexadecylmethylammonium salts, di((meth)acryloyloxyethyl)dodecylmethylammonium salts and (meth)acryloyloxyethyl(4-N-hexadecylpyridinylmethyl) succinate halides.

14. The method of claim 13, wherein the compound is selected from the group consisting of methacryloyloxydodecylpyridinium bromide (MDPB), diallyldimethylammonium chloride (DADMAC), methacryloyloxyhexedecylpyridinium chloride (MHPC), 4-hexadecyhnethacryloyloxyethylpyridinium chloride (HMPC),
methacryloyloxyethylhexadecylbipyridinium dichloride (MHBP),
methacryloyloxyoctadecyltrimethylphosphonium chloride (DMPC), methacryloyloxyoctadecyltriethylphosphonium acetate (OEPA), 4-methacryloyloxyethyldodecylpyridinium chloride (MEDP), di(4-vinylbenzyl)hexadecylmethylammonium methyl sulfate (VHMS), di(methacryloyloxyethyl)dodecylmethylammonium chloride (DDMC) and methacryloyloxyethyl(4-N-hexadecylpyridinylmethyl) succinate bromide (BMPS).

15. The method of claim 1, wherein applying the compound to the surface comprises spray application.

16. A method of preparing a medical implant having an anti-infective compound covalently bonded to a metal oxide present on the surface of the implant, the method comprising
applying an organic compound comprising a UV-photograftable moiety and an anti-infective moiety to the surface of the implant; and
applying UV light to the compound.

17. The method of claim 16, wherein the anti-infective moiety is a quaternary pyridinium, quaternary ammonium, or quaternary phosphonium moiety.

18. The method of claim 17, wherein the compound is MDPB.

19. An article prepared according to the method of claim 1.

20. An article prepared according to the method of claim 18.
